(19) United States
(12) Patent Application Publication
(10) Pub. No.: US 2025/0222054 A1
(43) Pub. Date: Jul. 10, 2025

(54) METHOD OF PREPARING PLANT EXTRACTS

(71) Applicant: Joseph Thomas FIDA, East Garafraxa (CA)

(72) Inventor: Joseph Thomas FIDA, East Garafraxa (CA)

(21) Appl. No.: 18/853,267

(22) PCT Filed: Mar. 31, 2023
(86) PCT No.: PCT/CA2023/050444

§ 371 Date: Oct. 1, 2024

Related U.S. Application Data
(60) Provisional application No. 63/326,487, filed on Apr. 1, 2022.

(51) Int. Cl. A61K 36/185; A61K 41/00

(52) U.S. Cl. CPC A61K 36/3482; A61K 41/00; A61K 2236/33; A61K 2236/53; A61K 2236/55

(57) ABSTRACT

Described is a method of reducing or eliminating chlorophyll content in a plant extract, while retaining certain other active components of said extract, by exposing said plant extract to a UV-C light source. Also described are extracts having lower levels of chlorophyll while retaining levels of certain other active components, such as cannabinoids and terpenes.

### FIELD OF THE INVENTION

The invention relates to the field of plant extract preparation, particularly to preparation of cannabis extracts having reduced chlorophyll content.

### BACKGROUND OF THE INVENTION

Plant extracts have become a popular nutritional, pharmaceutical, and recreational product. In particular, cannabis extracts, rich in terpenes and cannabinoid compounds such as THC and/or CBD have become an important commercial product.

Solvent extraction of cannabinoids from cannabis plant material is a common commercial process, providing cannabis extracts rich in desirable terpenes and cannabinoid compounds. Cannabinoids are notoriously hydrophobic, but dissolve in both alcohol and oil, so extractions are typically done using either oil-based or alcohol based solvents. Many different solvents are known, such as butanol, and methanol, with ethanol extraction being the most commonly used in large scale processes, since it provides a cannabinoid and terpene-rich extract in a solvent (ethanol) which can be ingested and is quite tolerable.

Because cannabis is a photosynthetic plant, it contains chlorophyll, which is unfortunately also highly soluble in alcohol, particularly ethanol. As a result, alcohol-based extraction processes typically provide an undesirable, darker colored, bitter tasting extract, due to the co-solubility of chlorophyll.

Certain methods are currently used to decrease the chlorophyll content of cannabis extracts. These are generally separated into methods utilized during the extraction, and post extraction methods. Cold extraction, where the extraction is performed at, for example, −35 to −40 degrees Celcius, results in less chlorophyll being solubilized. However, as can be appreciated, such cooling adds significantly to the cost of extraction, and may result in a different extraction profile for other ingredients, such as terpenes and cannabinoids. Post-extraction (“finishing”) methods include vacuum distillation, filtration including activated charcoal filtration, chromatographic separation using a filter or column, and separating out the chlorophyll using a hexane solubilization, where the ethanol extract is mixed with hexane; terpenes and cannabinoids typically remain in the aqueous (ethanol) layer, while chlorophyll is solubilized into the hexane.

Cannabis extracts with lower or no chlorophyll content have a more desirable golden color, and are much less bitter and more pleasant to ingest.

As can be appreciated, each of these methods for removing the chlorophyll adds complexity, cost, energy consumption, and, in some cases, environmental impact to the cannabis extract production process.

Accordingly, new methods for preparation of cannabis extracts with lower or no chlorophyll would be desirable.

### SUMMARY OF THE INVENTION

According to one aspect of the present invention is provided a method of manufacturing a plant extract from a plant material, comprising: adding a solvent to the plant material to form a solution; subjecting the solution to a UV-C light source; and collecting the liquid portion of the resultant solution as said plant extract.

In certain embodiments, the solvent comprises an alcohol, such as ethanol.

In certain embodiments, the plant material is a cannabis plant material.

In certain embodiments, the extract comprises cannabinoids and terpenes.

In certain embodiments, the extract contains less chlorophyll than an extract made without the step of subjecting the solution to the UV-C light source.

In certain embodiments, the extract contains no chlorophyll.

In certain embodiments, the method further comprises a finishing step, such as distillation, filtration, chromatographic separation, and hexane solubilization.

In certain embodiments, the UVC light source emits light preferentially at a wavelength of between 200 and 300 nm.

In certain embodiments, the UVC light source emits light preferentially at a wavelength of about 253 nm.

In certain embodiments, the UVC light source is a UV-C germicidal lamp of at least 25 W.

According to a further aspect of the present invention is provided a plant extract containing less than 10 mg/L chlorophyll.

According to a further aspect of the present invention is provided a plant extract manufactured by the method as herein described.

In certain embodiments, said plant material contains chlorophyll, wherein the plant extract contains less than 50%, 45%, 40%, 35%, 30%, 25%, 20%, 15%, 10%, or 5% of the chlorophyll of the plant material.

In certain embodiments, the plant material further comprises cannabinoids, wherein the plant extract contains more than 50%, 55%, 60%, 65%, 70%, 75%, 80%, 85%, 90%, or 95% of the cannabinoids of the plant material.

In certain embodiments, the plant material further comprises terpenes, wherein the plant extract contains more than 50%, 55%, 60%, 65%, 70%, 75%, 80%, 85%, 90%, or 95% of the terpenes of the plant material.

According to yet a further aspect of the present invention is provided a plant extract made from a plant material, said plant material containing chlorophyll, wherein the plant extract contains less than 50%, 45%, 40%, 35%, 30%, 25%, 20%, 15%, 10%, or 5% of the chlorophyll of the plant material.

In certain embodiments, the plant material further comprises cannabinoids, wherein the plant extract contains more than 50%, 55%, 60%, 65%, 70%, 75%, 80%, 85%, 90%, or 95% of the cannabinoids of the plant material.

In certain embodiments, the plant material further comprises terpenes, wherein the plant extract contains more than 50%, 55%, 60%, 65%, 70%, 75%, 80%, 85%, 90%, or 95% of the terpenes of the plant material.

In certain embodiments, the plant material is a cannabis plant material.

### BRIEF DESCRIPTION OF FIGURES

FIG. 1 shows the absorbance of UV-C light for chlorophyll standards, as well as treated and untreated samples.

FIG. 2 shows light absorption for cannabinoid extracts after UV-C treatment (Trial 1).

FIG. 3 shows light absorption for cannabinoid extracts after UV-C treatment (Trial 3).

FIG. 4 shows light absorption for cannabinoid extracts after UV-C treatment (Trial 4).

FIG. 5 shows light absorption for cannabinoid extracts after UV-C treatment (Trial 4).

FIG. 6 shows an untreated cannabinoid extract solution.

FIG. 7 shows a cannabinoid extract solution after 15 minutes of treatment.

FIG. 8 shows a cannabinoid extract solution after 30 minutes of treatment.

FIG. 9 shows a cannabinoid extract solution after 45 minutes of treatment.

FIG. 10 shows a cannabinoid extract solution after 60 minutes of treatment.

FIG. 11 shows a cannabinoid extract solution after 75 minutes of treatment.

FIG. 12 shows a cannabinoid extract solution after 90 minutes of treatment.

### DESCRIPTION

It has been found that chlorophyll can be removed from a cannabis extract through degradation of the chlorophyll using ultraviolet light. Specifically, an ultraviolet light in the UVC range (between about 200 nm to about 300 nm in wavelength, preferably between about 200 nm and 280 nm, more preferably about 254 nm) can be utilized to remove the unwanted chlorophyll from a cannabis extract.

UVC light is commonly used for disinfection, for example, in water purification systems. Accordingly, UVC lights are readily available and relatively inexpensive to purchase, use, and operate.

The amount of UVC light required can be measured in terms of intensity and exposure time. The intensity and exposure time needed will depend on a multitude of factors, including (1) the proximity of the light source to the extract; (2) the volume of extract being treated; (3) the concentration of chlorophyll in the extract, pre-treatment; (4) the desired concentration of chlorophyll in the extract, post-treatment; (5) the clarity or opaqueness of the extract and its particulate density; (6) the color of the extract; (7) the solvent used; (8) the concentration of terpenes and cannabinoids in the extract; (9) the material in which the extract is contained; as well as other factors. However, in general, the greater the intensity and duration of exposure of UVC light, the better in terms of reduction of chlorophyll in the extract.

We have surprisingly found that terpenes and cannabinoids, desirable in the cannabis extract post-treatment, are relatively unaffected by exposure to UVC light at intensities and durations that will degrade the chlorophyll. Although UVC light will eventually degrade the cannabinoids, with up to 45% of the cannabinoids being degraded after 180 minutes of treatment at 33.3 W/L of 253.7 nm light, chlorophyll degrades much more quickly, with the majority to all of the chlorophyll degraded after 30 to 60 minutes of the same treatment.

The exposure to UVC light may occur during the extraction process, or post-extraction, before, after, or instead of a traditional “finishing” step, and before solvent recovery. The degraded chlorophyll can remain in the extract, or be removed, through filtration or a traditional finishing step. The extract can be further treated to remove other impurities or to otherwise “finish” using traditional finishing steps such as distillation, filtration, chromatography, or the like. The extraction may be performed as a “cold” extraction, for example, at −35 to −40 degrees Celcius, at room temperature, or at any temperature therebetween.

The extraction can take place in any known extraction solvent, for example, an alcohol such as methanol, ethanol, isopropyl alcohol or butanol, or an oil such as vegetable oil. Preferably, the extraction solvent is clear and relatively colourless, to allow for improved transmission of the UVC light through the solvent.

The invention also relates to extracts made using said UV-C treatment process; the extracts are notably low in chlorophyll, which makes them more palatable, less bitter, and of a pleasing color.

Although the present examples are directed to the manufacture of cannabis extract (and the extract manufactured therefrom), it would be suitable for removing chlorophyll from any plant extract, particularly plant extracts made using similar techniques and/or solvents. Plant extracts are quite common for a wide variety of plants, and a wide variety of uses, including as food ingredients, perfumes, pharmaceuticals, nutraceuticals, and homeopathic medicines.

The present method may make an extract containing greater than 95%, 90%, 85%, 80%, 75%, 70%, 65%, 60%, 55%, or 50% of the cannabinoids and terpenes of the plant material from which the extract is made, while containing less than 50%, 45%, 40%, 35%, 30%, 25%, 20%, 15%, 10%, or 5% of the chlorophyll of the plant material from which the extract is made.

### Example 1: Cannabis Extraction

Approximately 5 g of cannabis plant material, including flower buds and leaves, were dried and pulverized into a relatively fine powder. To the powder was added 360 mL of 70% food grade ethanol. The resultant suspension/mixture was stirred using a magnetic stir bar for 10 minutes in an open top beaker. Sample powder was allowed to settle, and the clear sample solution was decanted. Alternatively, the mixture was passed through a strainer and/or a chinois to separate the alcohol extract from the plant matter. The extract (containing both cannabinoids and chlorophyll) was placed in a 304ss closed vessel with a submerged UVC light bulb (12 W 253 nm UV germicidal lamp from Suyooulin, purchased on Amazon.com), shielded by a quarts sheath. The light was turned on and samples were taken at regular timed intervals (every 15 minutes); the vessel was gently agitated before every sample was taken.

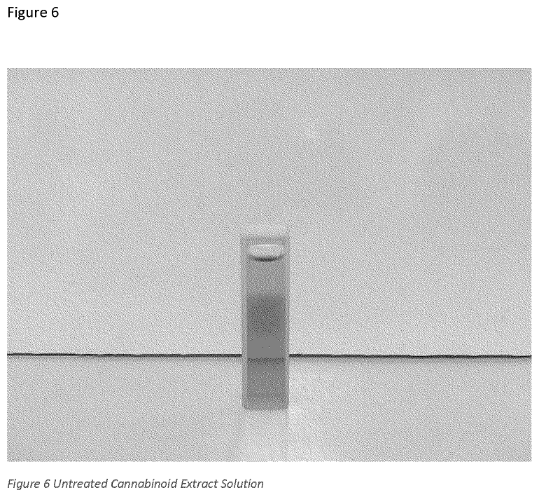

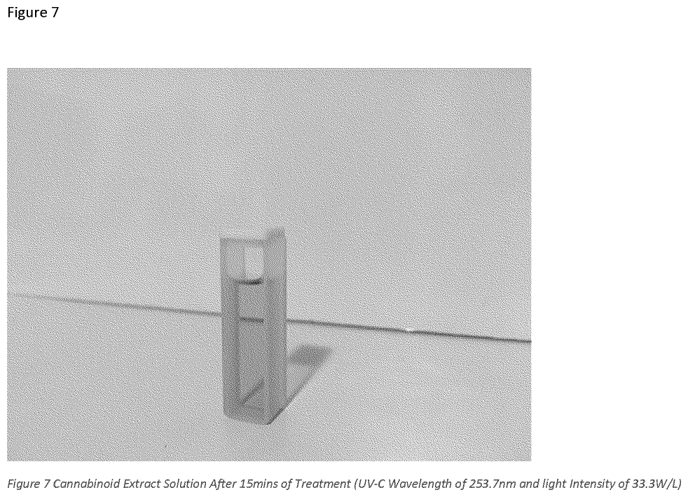

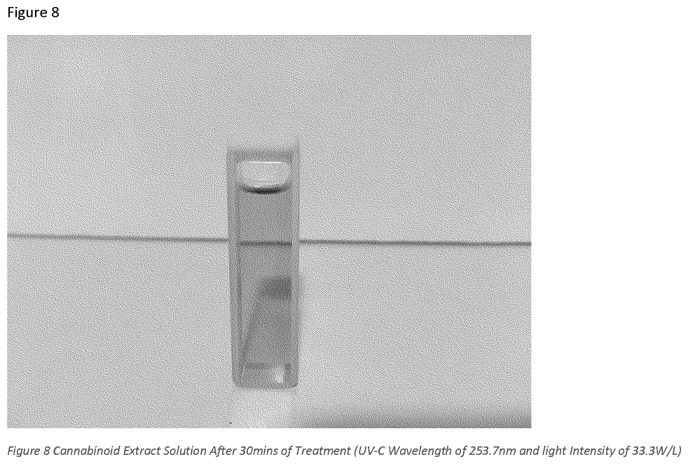

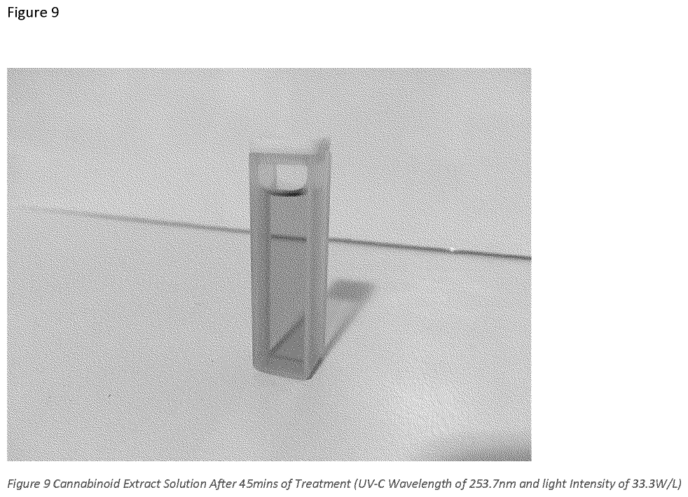

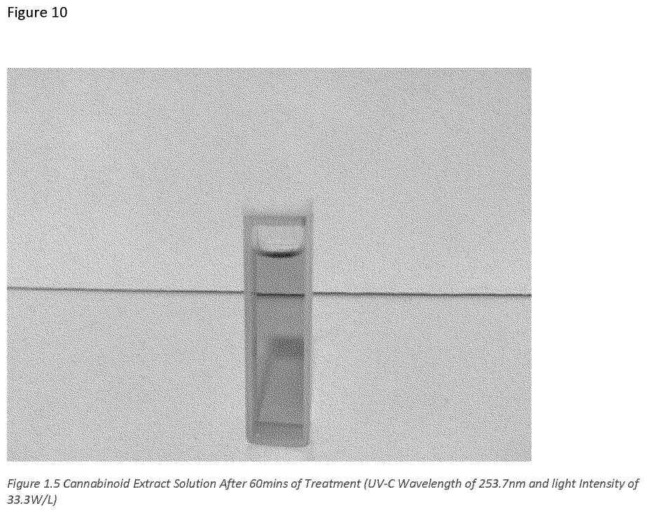

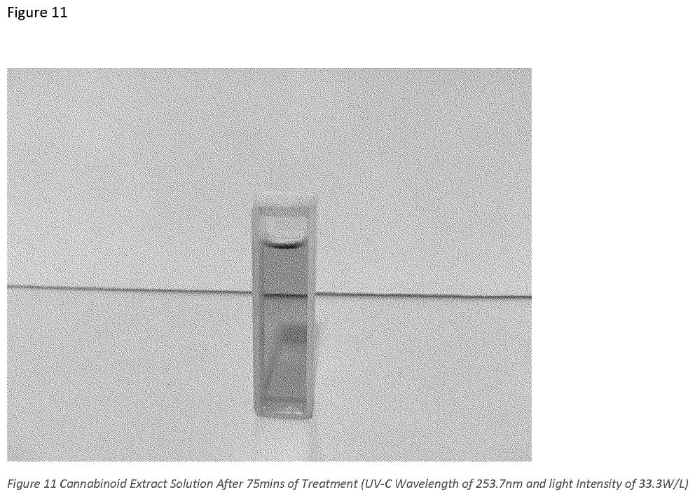

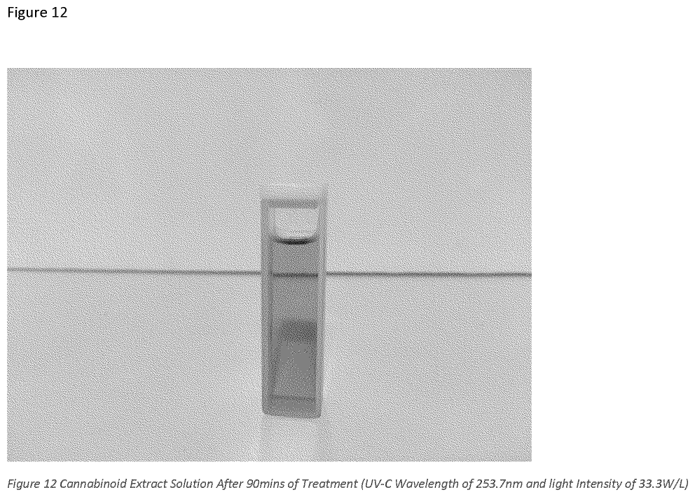

The resultant UVC-treated cannabis extract (in alcohol) was much lighter color, and much less bitter tasting, than a (non-UVC treated) control extract, after 15-75 minutes of UVC treatment (see FIGS. 6-12, which show pictures of cannabis extract, untreated (FIG. 6) or after 15, 30, 45, 60, 75 and 90 minutes of UV-C treatment at 253.7 nm at a light intensity of 33.3 W/L (FIGS. 7, 8, 9, 10, 11, and 12, respectively). The extract can be seen with the naked eye to be gradually but increasingly less green, and of a lighter, yellower color as the time of UV-C treatment increases.

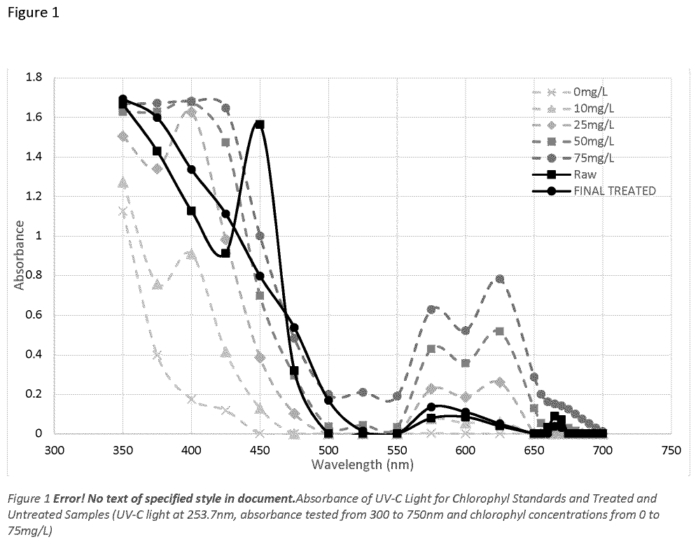

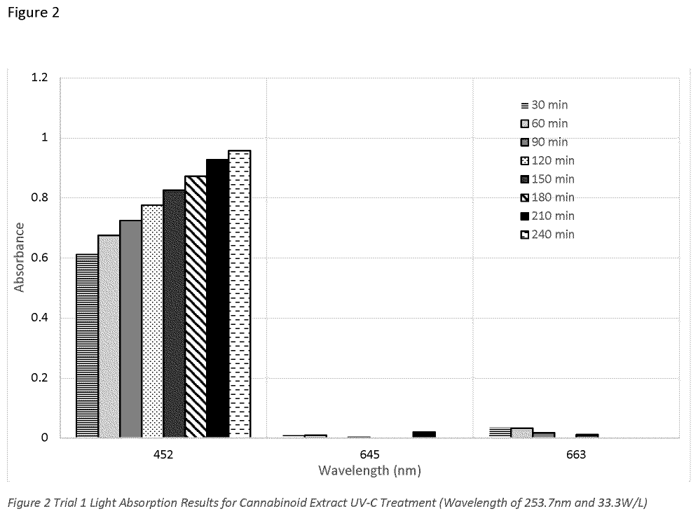

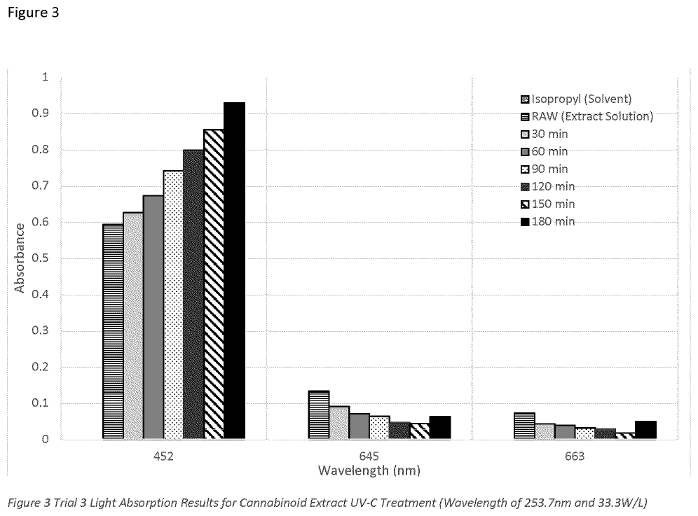

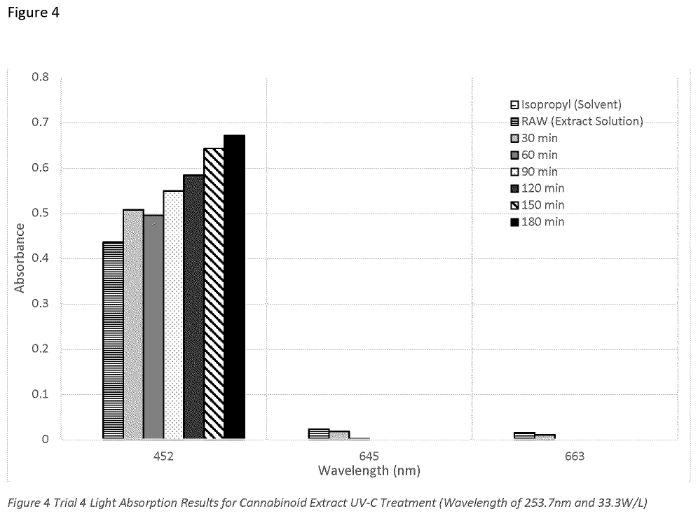

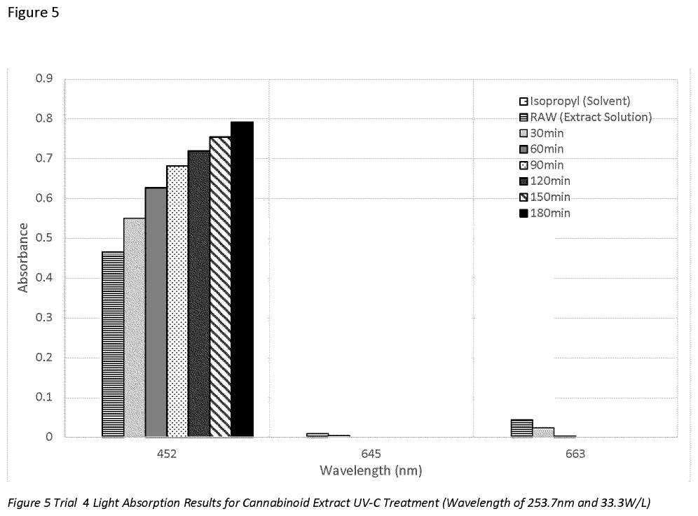

Both control and UVC-treated extracts were found to contain similar amounts of terpenes and cannibinoids, with the UVC-treated cannabis extract containing significantly less chlorophyll A and B, as confirmed both by ultra high-performance liquid chromatography (UHPLC), as well as measurement of absorbance of diluted standards at 430 nm, 662 nm, 453 nm and 665 nm using a spectrophotometer (FIGS. 1-5). FIG. 1 shows raw (untreated) as well as final treated (treated with 60 minutes of UV-C light at 253.7 nm and 33.3 W/L), as compared to Omg/L, 10 mg/L, 25 mg/L, 50 mg/L, and 75 mg/L standardized controls for chlorophyll. FIG. 2 shows light absorption measurements at 452, 645 and 663 nm for cannabinoid extract after treatment (30, 60, 90, 120, 150, 180, 210 or 240 minutes of UV-C light at 253.7 nm and 33.3 W/L), indicating a significant and steady increase in absorbance at 452 nm with increases in treatment time. FIGS. 3, 4 and 5 show similar results, and an indication of decreased absorbance at 645 and 663 nm as treatment time increased, in repeated experiments.

1. A method of manufacturing a plant extract from a plant material, comprising:
adding a solvent to the plant material to form a solution;
subjecting the solution to a UV-C light source;
collecting the liquid portion of the resultant solution as said plant extract.

2. The method of claim 1 wherein the solvent comprises an alcohol.

3. The method of claim 2 wherein the alcohol comprises ethanol.

4. The method of claim 1 wherein the plant material is a cannabis plant material.

5. The method of claim 4 wherein the extract comprises cannabinoids and terpenes.

6. The method of claim 4 wherein the extract contains less chlorophyll than an extract made without the step of subjecting the solution to the UV-C light source.

7. The method of claim 4 wherein the extract contains no chlorophyll.

8. The method of claim 1 further comprising a finishing step selected from the group consisting of distillation, filtration, chromatographic separation, and hexane solubilization.

9. (canceled)

10. The method of claim 1 wherein the UVC light source emits light preferentially at a wavelength of between 200 and 300 nm.

11. The method of claim 10 wherein the UVC light source emits light preferentially at a wavelength of about 253 nm.

12. The method of claim 1 wherein the UVC light source is a UV-C germicidal lamp of at least 25 W.

13. A plant extract containing less than 10 mg/L chlorophyll.

14. A plant extract manufactured by the method of claim 1.

15. The plant extract of claim 14 wherein said plant material contains chlorophyll, wherein the plant extract contains less than 50%, 45%, 40%, 35%, 30%, 25%, 20%, 15%, 10%, or 5% of the chlorophyll of the plant material.

16. The plant extract of claim 15, wherein the plant material further comprises cannabinoids, wherein the plant extract contains more than 50%, 55%, 60%, 65%, 70%, 75%, 80%, 85%, 90%, or 95% of the cannabinoids of the plant material.

17. The plant extract of claim 15, wherein the plant material further comprises terpenes, wherein the plant extract contains more than 50%, 55%, 60%, 65%, 70%, 75%, 80%, 85%, 90%, or 95% of the terpenes of the plant material.

18. A plant extract made from a plant material, said plant material containing chlorophyll, wherein the plant extract contains less than 50%, 45%, 40%, 35%, 30%, 25%, 20%, 15%, 10%, or 5% of the chlorophyll of the plant material.

19. The plant extract of claim 18, wherein the plant material further comprises cannabinoids, wherein the plant extract contains more than 50%, 55%, 60%, 65%, 70%, 75%, 80%, 85%, 90%, or 95% of the cannabinoids of the plant material.

20. The plant extract of claim 18, wherein the plant material further comprises terpenes, wherein the plant extract contains more than 50%, 55%, 60%, 65%, 70%, 75%, 80%, 85%, 90%, or 95% of the terpenes of the plant material.

21. The plant extract of claim 19, wherein the plant material is a cannabis plant material.
